(19) United States
(12) Patent Application Publication
(10) Pub. No.: US 2026/0137910 A1
(43) Pub. Date: May 21, 2026

(54) NEEDLESTICK INJURY PREVENTION DEVICE FOR MEDICAL PUNCTURE DEVICE, AND MEDICAL INJECTION AND INFUSION DEVICE

(71) Applicant: SUZHOU LINHWA MEDICAL DEVICES CO., LTD., Suzhou (CN)

(72) Inventor: Changning NIE, Suzhou (CN)

(21) Appl. No.: 19/452,948

(22) Filed: Jan. 20, 2026

Related U.S. Application Data
(63) Continuation of application No. PCT/CN2023/124733, filed on Oct. 16, 2023.

(30) Foreign Application Priority Data
Aug. 31, 2023 (CN) 202311114285.7

(51) Int. Cl. A61M 25/06

(52) U.S. Cl. CPC A61M 25/0618; A61M 25/0693

(57) ABSTRACT

A needlestick injury prevention device for a medical puncture device and a medical injection and infusion device are disclosed. The needlestick injury prevention device for the medical puncture device includes a sheath, a puncture needle, and a retainer elastic piece, wherein the retainer elastic piece is arranged in the sheath, and the puncture needle is arranged between the retainer elastic piece and the sheath. The device adopts an integrated molded retainer elastic piece which, under directional guidance of a base and in coordination with the sheath, cooperates with a blood flashback groove of a tube of the puncture needle to achieve rapid activation covering a full 360-degree range.

### CROSS-REFERENCE TO RELATED APPLICATIONS

The present application is a continuation of International Application No. PCT/CN2023/124733, with an international filing date of October 16, 2023, which is based upon and claims priority to Chinese Patent Application No. 202311114285.7, filed on August 31, 2023, the entire contents of all of which are incorporated herein by reference.

### TECHNICAL FIELD

The present disclosure relates to the technical field of medical instruments, and in particular, to a needlestick injury prevention device for a medical puncture device, and a medical injection and infusion device.

### BACKGROUND

Globally, the issue of occupational exposure and infection among healthcare workers due to unsafe infusion practices has always been prevalent. Medical personnel face the risk of sharp instrument injuries when using puncture sets. Safe infusion is the fundamental pathway and effective measure to prevent the transmission of infections and ensure both patient safety and occupational safety for healthcare workers. In recent years, with the growing awareness of safety protection, along with continuous development in technical means and clinical medical exploration and research, sharp instrument injury protection devices have been integrated as components into infusion device products (such as hypodermic needles, infusion needles, indwelling needles, and vascular puncture needles), providing new pathways and methods for safe infusion.

Indwelling needles commonly use a catheter over needle (CON) catheterization method, referred to as a CON catheterization method hereinafter. This catheterization method is increasingly used and often combined with the Seldinger technique. For example, patent CN105611962B discloses a catheter insertion device that allows for one-handed insertion of a catheter into a blood vessel of a patient. In this patent, a catheter, a needle tube, and a guidewire that are originally required are integrated into a single device and are arranged concentrically with the catheter outermost, the needle tube intermediate, and the guidewire innermost. Using the Seldinger technique, the puncture needle and the guidewire are replaced, and the catheter is implanted. The guidewire needs to pass through the interior of the needle tube of this device, and the inner diameter of the needle tube needs to precisely match the outer diameter of the guidewire, especially for small-sized needle tubes. To avoid the guidewire from being unable to pass through, which affects the performance of the device, the precision required for the fit between the inner diameter of the needle tube and the outer diameter of the guidewire needs to be further improved.

Currently, the processing mode for sharp instrument injury protection devices for infusion device products generally involves pre-fabricating local deformation features such as 'flattened sections' and 'indentations' on the needle tube, and then providing a 'stop plate' that matches the outer diameter of the needle tube and a compressed 'protective elastic piece'. The working principle of this structural form is as follows: after the device completes its intended use, the needle tube is pulled during the removal process. When a locally deformed position on the needle tube reaches a fixed position of the 'stop plate', the compressed 'protective elastic piece', no longer restrained by the compressive force from the needle tube, releases its elastic energy, quickly restoring to its original shape and covering a needle tip to activate the protective device for protection. For example, in the utility model patent with the grant announcement No. CN218944044U, entitled “Needle Tip Locking Device for Puncture or Medication Infusion Needles”, in the operational state, a needle tip locking assembly is connected to the needle tube through a through hole in a fixed plate. When a needle handle is pulled to withdraw the needle tube, a needle tip at a front end of the needle tube retracts into the needle tip locking assembly and is wrapped within a cavity formed by the engagement of a first needle tip locking cover and a second needle tip locking cover in the needle tip locking assembly. Due to a pre-set flattened area on the needle tube that matches the through hole in the fixed plate, the needle tip locking assembly is eventually pulled out with the needle tube, thereby shielding the needle tip of the puncture or infusion needle to prevent injury to an operator when not in use. For another example, in the utility model patent with the grant announcement No. CN203577057U, entitled “Needlestick injury prevention Venous Indwelling Needle”, a needlestick-prevention safety device is provided on a needle tube between a needle hub and a catheter hub. The needle tube is provided with an indentation near a needle tip. When the needle hub and the needle tube are pulled out together, a safety clip in the needlestick injury prevention safety device blocks a central hole 3 in an end A of the needlestick injury prevention safety device, and a metal plate at an end B of the safety device locks into the indentation on the needle tube. In the aforementioned patents, the needle hub needs to be pre-processed with flattened areas or indentations. This pre-processing method, achieved by locally deforming the needle tube, is classified as “damage-based protection”. That is, by locally processing the needle tube, which causes local deformation in the inner and outer diameters of the needle tube, the inner diameter of the deformed part of the needle tube becomes smaller, and the outer diameter becomes larger, compromising and affecting the shape, size, and functional application of components that are assembled with the needle tube, and further resulting in some functions being unachievable. For example, when a guidewire needs to pass through a vascular puncture needle, if it encounters a locally deformed section of the needle tube, the reduced inner diameter of the needle tube at that point can easily cause blockage and make passage impossible. Additionally, the manufacturing process for each component in this structure is relatively complex (such as controlling the stamping force for creating flattened sections or indentations), leading to high manufacturing costs. Moreover, such protective device, which relies on creating stop positions, is prone to failure due to misalignment or instability between the stop components (such as the stop plate) and the needle tip.

To solve the problems associated with the aforementioned “damage-based protection”, existing technical solutions, for example, the utility model patent with the grant announcement No. CN216933402U, entitled “Needlestick injury prevention Protective Device” (hereinafter referred to as Reference 1), involves providing an elastic stopper inside a housing. A needle hole and a strip-shaped slot that communicates with the needle hole and has a width smaller than the outer diameter of the needle hole are provided in the elastic stopper. A radial slot is provided in a side face of a front end of the needle tube. In an ideal state, when the needle tube is withdrawn, the radial slot passes the needle hole. Due to the reduced outer diameter of the needle tube, the elastic stopper displaces, causing the radial slot of the needle tube to move into the strip-shaped slot. Since the width of the strip-shaped slot is smaller than the outer diameter of the needle hole, the needle tube is less likely to disengage from the strip-shaped slot, and hence, the needlestick injury prevention effect can be achieved.

However, the structure also has the following issues in actual operation:

As shown in FIG. 1, an automatic self-rebounding section of the elastic stopper always presses against an inner wall of the housing, and the strip-shaped slot presses against the needle tube. This creates a reactive force with a “support arm” structure on the other side of the automatic self-rebounding section of the elastic stopper, forming a complex resultant force system. This results in significant axial movement friction resistance between the needle tube and the elastic stopper as well as the housing, which can easily lead to problems such as jamming. At the same time, since both sides of the elastic stopper always press against the inner wall of the housing, the housing itself cannot achieve relative displacement with the needle tube. Therefore, under the premise that the needle tube remains stationary, it is impossible to achieve relative retraction of the needle tube by pushing the housing until it covers the needle tip. The protection against needlestick injuries can only be achieved through the single operation of axially pulling the needle tube.

Limited by the shapes and structures of the strip-shaped slot (the width of the strip-shaped slot is smaller than the diameter of the needle hole) in the elastic stopper and the radial slot in the needle tube, only “directional displacement to activate protection” can be achieved. That is, protection is only activated when “a plane where the radial slot is located and a length direction of the strip-shaped slot are in the same plane. Hence, engagement can be achieved by making the radial slot and the strip-shaped slot come into contact” (as described in paragraph [0034] of Reference 1). This cannot achieve 360-degree displacement to activate protection around the circumference of the needle tube.

Since the automatic self-rebounding section of the elastic stopper and the “support arm” structure form an intersecting force, the reactive force generated by the “support arm” structure, influenced by the structural configuration, will counteract an inclined force produced by compressive deformation of the automatic self-rebounding section. Therefore, the deformation speed of the automatic self-rebounding section is slowed down. At the moment of needle tube removal, if the response speed of the deformation of the automatic self-rebounding section is less than the withdrawal speed of the needle tube, the radial slot of the needle tube will pass over the needle hole of the elastic stopper, leading to protection failure.

The protective device relies on the deformation force generated by the compressive deformation of the automatic self-rebounding section of the elastic stopper to undergo displacement and locking protection when passing the radial slot of the needle tube. This requires a fast response speed of the elastic stopper to elastic deformation. Therefore, when such structure is applied to puncture needle tube products with very limited fitting space, the elastic stopper needs to be made of a material with good elastic deformation properties. At the same time, to improve the sensitivity of the structure, reducing the thickness of the material is needed. The material is easily deformed with the thickness reduced, and the presence of the strip-shaped slot increases the void area, further weakening the structural support performance and structural strength. Relying solely on the small structural strength of the strip-shaped slot to achieve locking and blocking, when the needle withdrawal force is relatively great, the strip-shaped slot is likely to be directly stretched open, allowing it to widen and let the needle tube pass through, resulting in failure.

### SUMMARY

Therefore, to solve the aforementioned issues, the present disclosure provides a needlestick injury prevention device for a medical puncture device.

The present disclosure is implemented through the following technical solution.

A needlestick injury prevention device for a medical puncture device includes a sheath, a puncture needle, and a retainer elastic piece, wherein the retainer elastic piece is arranged in the sheath, and the puncture needle is arranged between the retainer elastic piece and the sheath.

The sheath includes a main body and a base arranged at the bottom of the main body. The base is provided with a limiting step on a first side. The puncture needle includes a hollow tube, a needle tip located at one end of the tube, and a blood flashback groove provided in an outer periphery of the tube. The retainer elastic piece includes a fixed portion, and a bottom hole is provided in the fixed portion. A support arm is arranged on a first side of the fixed portion, and a resilient arm is arranged on a second side. The support arm includes a first side arm that bends upward from one side of the fixed portion and a guide portion that bends from the top of the first side arm and is parallel to the fixed portion. A strip-shaped slot is provided in the support art and communicates with the first side arm and the guide portion. An open guide hole is further provided in the guide portion and disposed in the end of the strip-shaped slot. An opening is formed in an end of the open guide hole that connects to the strip-shaped slot, and the open guide hole is coaxially arranged with the bottom hole.

A width V of the tube of the puncture needle at the position of the blood flashback groove is less than a width V of the opening of the open guide hole, the width V of the opening of the open guide hole is less than the outer diameter of the tube of the puncture needle, and the outer diameter of the tube of the puncture needle is less than the width of the strip-shaped slot.

In an initial state, the first side of the fixed portion is placed flat on the limiting step, the tube of the puncture needle passes coaxially through the bottom hole and the open guide hole, the blood flashback groove is located at the top of the open guide hole, and the resilient arm elastically abuts against the tube of the puncture needle.

In an activated state, the blood flashback groove in the puncture needle retreats to a height of the open guide hole and moves from the opening of the open guide hole to the strip-shaped slot, the retainer elastic piece tilts to the second side, the fixed portion moves away from the surface of the limiting step, the bottom hole tilts with the fixed portion, a wall of the bottom hole exerts a cross torque on the outer periphery of the tube of the puncture needle to hold the tube tightly, and the resilient arm still abuts against the tube of the puncture needle and increases the torque at a contact point between the two as the retainer elastic piece tilts.

Preferably, the opening between the open guide hole and the strip-shaped slot is provided with arc-shaped transition surfaces symmetrically arranged on its opposite edges.

Preferably, two reinforcing arms are arranged at two ends of the fixed portion between the support arm and the resilient arm. Each of the reinforcing arms has a greater thickness than other positions of the fixed portion. A first side shoulder is arranged at one side of each reinforcing arm close to the support arm, and a second side shoulder is arranged at one side of each reinforcing arm close to the resilient arm. The fixed portion, the support arm, the resilient arm, and the reinforcing arms are integrally formed.

Preferably, the resilient arm is generally S-shaped, including a second side arm that bends upward from one side of the fixed portion. The top of the second side arm bends to the second side to form a positioning portion. In the initial state, a junction between the second side arm and the positioning portion elastically abuts against the outer periphery of the tube of the puncture needle.

Preferably, the second side arm includes a first connecting portion connected to the positioning portion, and a second connecting portion connected to the fixed portion. A necked portion is arranged between the first connecting portion and the second connecting portion and gradually narrows from the first connecting portion to the second connecting portion. A width of the first connecting portion is equal to a maximum width of the necked portion, and a width of the second connecting portion is equal to a minimum width of the necked portion.

Preferably, the first side arm tilts toward the second side, and the second side arm tilts toward the first side. The connection point of the second side arm is located at an intersection between a plane where a side edge of the first side is located and the bottom hole.

Preferably, two pressing posts are arranged on an inner wall of the sheath at the second side. A distance between the two pressing posts is equal to a distance between the two reinforcing arms, and the two pressing posts are respectively arranged on top of the second side shoulders of the two reinforcing arms.

Preferably, the base is provided with locking seats on both sides of the limiting step. A limiting groove is provided at the top of each locking seat, and a latch is arranged at the outer periphery of the locking seat. A catch matching the latch is provided in the outer periphery of the sheath.

Preferably, a first through hole is provided in the base. A sleeve is arranged at the top of the sheath, and a puncture channel is provided in the sleeve. The first through hole and the puncture channel are coaxially arranged.

A medical injection and infusion device includes the aforementioned needlestick injury prevention device for the medical puncture device.

The main advantageous effects of the technical solution of the present disclosure are as follows.

When the puncture needle needs to activate locking, the retainer elastic piece is maintained tilted. At this time, an inclined cross torque generated at the position of the bottom hole causes the tube to be tightly limited and protected, changing the bottom hole from a “straight guiding sliding mode” to a “tilted cross torque clamping and locking mode.” In the tilted cross torque clamping and locking mode, the bottom hole presses and locks the tube along its outer periphery in a 360-degree rotation and maintains its position. Regardless of whether the puncture needle continues to be pushed or pulled axially, the bottom hole always remains, in the tilted cross torque clamping and locking mode, pressing and locking the tube and maintaining its position. When an external force applied is greater, a self-locking retention force also increases. At this point, the needle tip of the puncture needle also retracts into the sheath, preventing the needle tip from being exposed.

2. The opening width of the open guide hole is less than the outer diameter of the tube of the puncture needle. Therefore, in the initial state, the tube of the puncture needle can be stably fixed in the open guide hole and cannot move from the opening of the open guide hole to the strip-shaped slot. When the position of the blood flashback groove moves to the open guide hole, the width of the tube at this position is less than the width of the opening of the open guide hole, allowing the tube to pass through the opening in a 360 -degree rotation and move into the strip-shaped slot without the need for “directional activation,” ensuring 360-degree tilt displacement for limited protection without blind spots, thereby greatly enhancing the flexibility and safety of the device.

3. The resilient arm is deformed by compression to generate a deformation force when installed with the puncture needle, and the deformation force is then applied at the point of contact with the tube. In the initial state, through guidance of the structural shape and under the limiting action of the bottom hole and the open guide hole, an inclined deflection elastic retention force is generated. In the activated state, the relative movement of the tube is achieved by either pulling the puncture needle backward or pushing the sleeve while the puncture needle remains stationary. When the guide groove moves into alignment with the open guide hole, the open guide hole loses its coaxial constraint on the puncture needle. The elastic retention force released from the resilient arm then rapidly propels the tube toward the strip-shaped slot and causes the retainer elastic piece to tilt and deflect, thereby preventing any jamming.

4. The resilient arm is provided with a necked portion gradually narrowing between the first connecting portion and the second connecting portion, which can further enhance the elasticity sensitivity of the resilient arm. When the puncture needle is installed in the retainer elastic piece, the resilient arm is expanded by the puncture needle and compressed to generate a deformation force. A reactive force is then exerted on the puncture needle, pushing the puncture needle towards the strip-shaped slot. The width of the strip-shaped slot is greater than the outer diameter of the tube of the puncture needle, which causes no cross and staggered interference during the inclined displacement, thereby improving the sensitivity and force precision of the overall structure.

5. Two reinforcing arms are arranged at both ends of the fixed portion of the retainer elastic piece between the support arm and the resilient arm. The fixed portion, the support arm, the resilient arm, and the reinforcing arms are integrally formed. The reinforcing arms can increase the strength of the bottom of the retainer elastic piece. Through such locally reinforced structure, the ability to resist an external force and prevent failure is significantly increased while reducing the overall weight and volume of the retainer elastic piece.

6. The first side of the base is provided with a limiting step. In the initial state, the first side of the fixed portion is placed flat on the limiting step, while the second side of the base is not provided with a limiting step, thereby providing a space for the retainer elastic piece to tilt towards the second side later on. At the same time, pressing posts are arranged at the inner wall of the sheath corresponding to the top portions of the two second side shoulders of the reinforcing arms. When the retainer elastic piece tilts, the pressing posts arranged on the inner wall of the sheath can also abut against the two second side shoulders of the reinforcing arms even if the puncture needle is pushed outward, preventing the needle tip of the puncture needle from being exposed, thereby providing a secondary protective structure.

### BRIEF DESCRIPTION OF THE DRAWINGS

FIG. 1 is a schematic diagram of a force state of each structure in Reference 1 in the background art.

FIG. 2 is a structural schematic diagram (partially cut) of a needlestick injury prevention device for a medical puncture device in an initial state.

FIG. 3 is a structural schematic diagram (partially cut) of the needlestick injury prevention device for the medical puncture device in an activated state.

FIG. 4 is a sectional view of the needlestick injury prevention device for the medical puncture device in the initial state.

FIG. 5 is a sectional view of the needlestick injury prevention device for the medical puncture device in the activated state.

FIG. 6 is an enlarged view of part A in FIG. 5.

FIG. 7 is a structural schematic diagram of a base.

FIG. 8 is a schematic diagram of a force state between a bottom hole of a retainer elastic piece and a tube of a puncture needle in the activated state.

FIG. 9 is a structural schematic diagram illustrating a state of a sheath.

FIG. 10 is a three-dimensional view of the retainer elastic piece.

FIG. 11 is a side view of a second side of the retainer elastic piece.

FIG. 12 is a schematic diagram illustrating comparison of the width between the tube of the puncture needle at the position of a blood flashback groove and the opening of the open guide hole.

FIG. 13 is a schematic diagram of a disassembled state of each component of the needlestick injury prevention device for the medical puncture device.

### DETAILED DESCRIPTION OF THE EMBODIMENTS

To present the objectives, advantages and features of the present disclosure more clearly and in greater detail, the following preferred embodiments will be illustrated and explained through non-limiting descriptions. These embodiments are merely typical examples applying the technical solution of the present disclosure , and any technical solutions formed by equivalent substitutions or transformations fall within the scope of protection of the present disclosure.

As also stated in the description of the solution, it should be noted that orientations or positional relationships indicated by terms such as “central,” “upper,” “lower,” “left,” “right,” “front,” “rear,” “inner” and “outer” are based on the orientations or positional relationships shown in the accompanying drawings. These terms are used solely for convenience and simplification of description, and do not indicate or imply that the devices or elements referred to must have specific orientations, be constructed, or operated in specific orientations. Therefore, they should not be understood as limitations on the present disclosure.

Furthermore, the terms “first” and “second” in the solution are used for descriptive purposes only and should not be understood as indicating or implying an order of importance or suggesting a specific number of the technical features shown. Therefore, features qualified with “first” or “second” can explicitly or implicitly include one or more of such features. In the present disclosure, the term “a plurality of” means two or more, unless otherwise specifically defined.

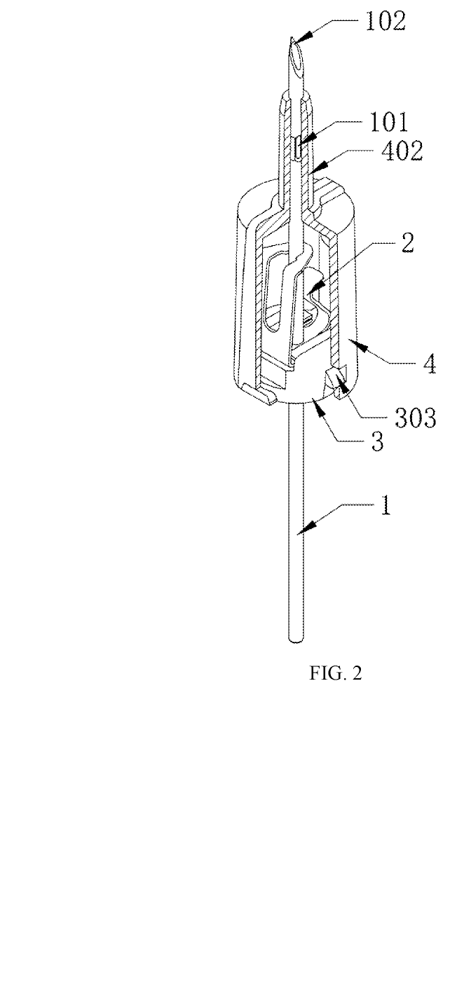

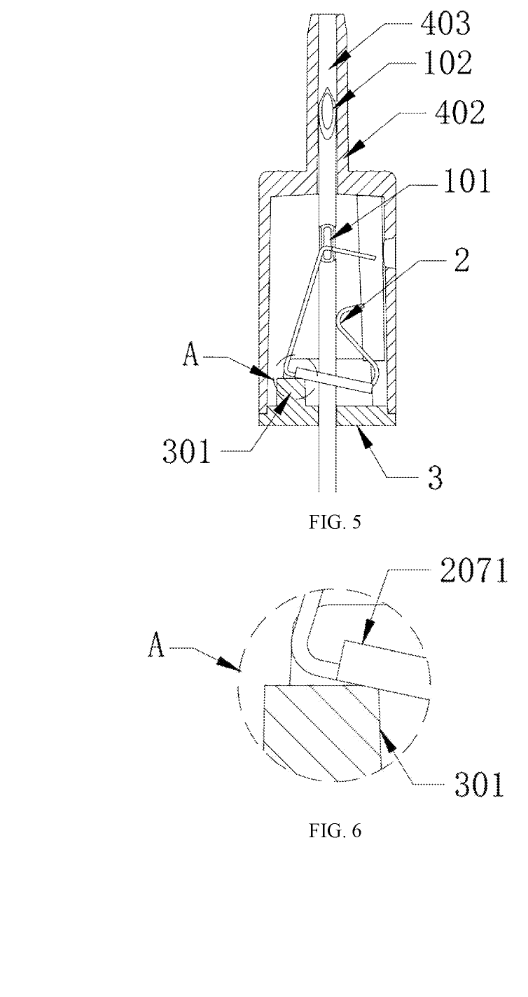

The present disclosure discloses a needlestick injury prevention device for a medical puncture device, as shown in FIGS. 2, 5, and 13, including a sheath 4, a puncture needle, and a retainer elastic piece 2. The retainer elastic piece is arranged in the sheath 4, and the puncture needle is arranged between the retainer elastic piece 2 and the sheath 4.

The sheath 4 includes a main body and a base 3 arranged at the bottom of the main body. A limiting step 301 is arranged on a first side of the base 3. In some embodiments, the limiting step 301 is a limiting block arranged on an upper surface of the base 3 in a protruding manner. The top of the limiting block is a plane, and the limiting is used for positioning the retainer elastic piece 2 during assembly.

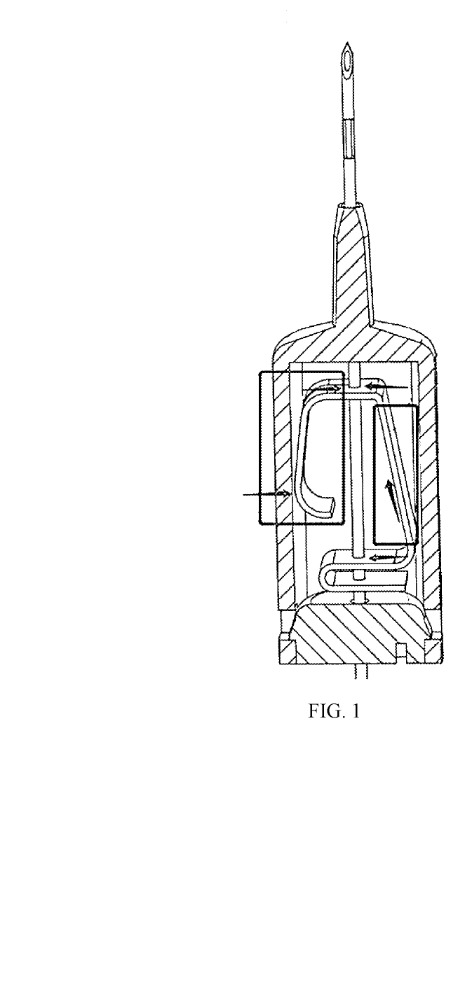

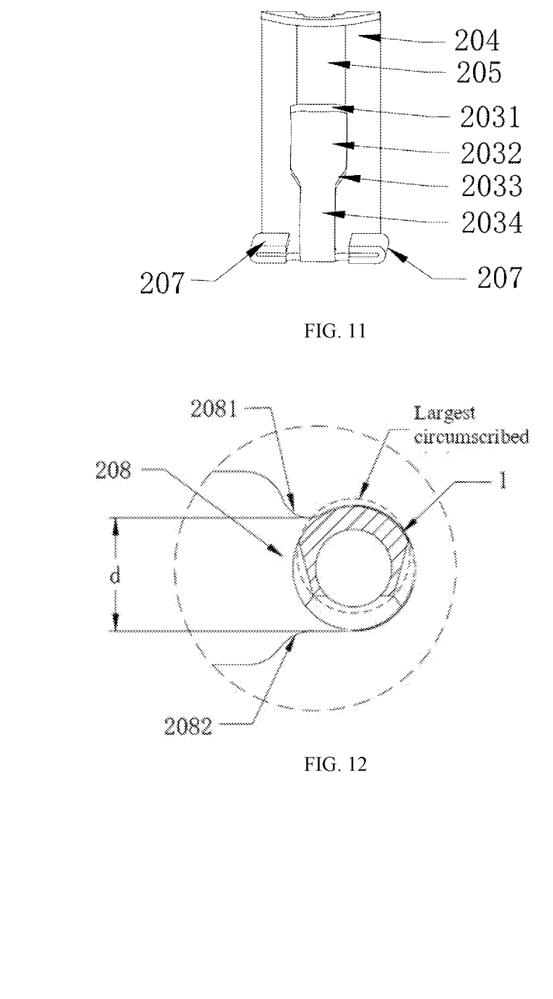

The puncture needle includes a hollow tube 1, a needle tip 102 located at one end of the tube 1, and a blood flashback groove 101 provided in the outer periphery of the tube 1. In some embodiments, a guidewire can penetrate through the interior of the puncture needle, and the puncture needle can also be used for injection or infusion. As shown in FIGS. 1 and 11, the retainer elastic piece 2 includes a fixed portion 201. A bottom hole 202 is provided in the fixed portion 201. A support arm 204 is arranged on a first side of the fixed portion 201, and an resilient arm 203 is arranged on a second side. The support arm 204 includes a first side arm that bends upward from one side of the fixed portion 201 and a guide portion 209 that bends from the top of the first side arm and is parallel to the fixed portion 201. A strip-shaped slot 205 is provided in the support arm 204 and communicates with the first side arm and the guide portion 209. An open guide hole 206 is further formed in the guide portion 209 and provided in an end of the strip-shaped slot 205. An opening 208 is formed in one end of the open guide hole 206 that connects to the strip-shaped slot 205, and the open guide hole 206 is coaxially arranged with the bottom hole 202.

The width of the tube 1 of the puncture needle at the position of the blood flashback groove 101 is less than the width of the opening 208 of the open guide hole 206, the width of the opening 208 of the open guide hole 206 is less than the outer diameter of the tube 1 of the puncture needle, and the outer diameter of the tube 1 of the puncture needle is less than the width of the strip-shaped slot 205.

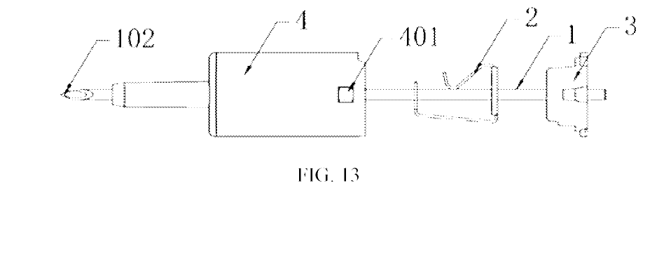

As shown in FIG. 12, the width of the tube 1 of the puncture needle at the position of the blood flashback groove 101 is less than the width of the opening 208 of the open guide hole 206, meaning that the outer diameter of the largest circumscribed circle formed by connecting points at each cut position of the tube at the position of the blood flashback groove is less than the width d of the opening 208 of the open guide hole 206. The shape of the blood flashback groove can be adjusted as needed, which is not further elaborated here.

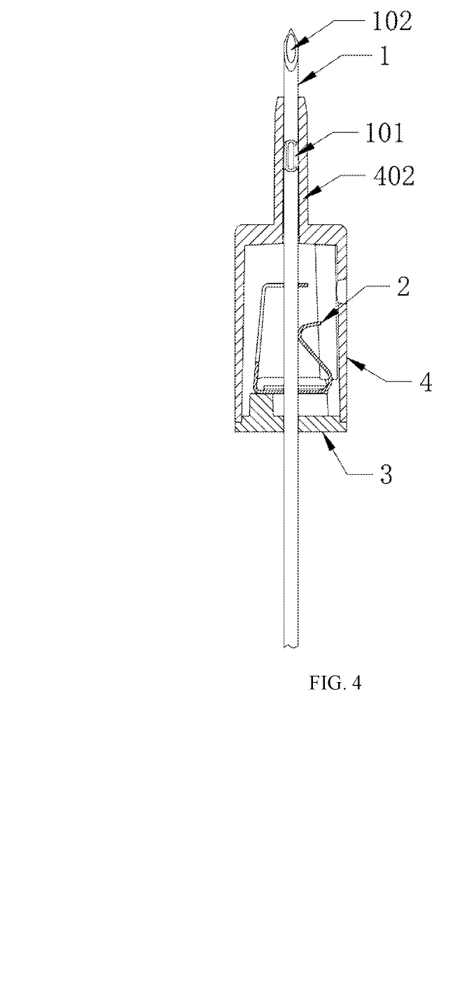

As shown in FIGS. 2 and 4, in the initial state, the first side of the fixed portion 201 is placed flat on the limiting step 301, the tube 1 of the puncture needle passes coaxially through the bottom hole 202 and the open guide hole 206, the blood flashback groove 101 is located at the top of the open guide hole 206, and the resilient arm 203 elastically abuts against the tube 1 of the puncture needle.

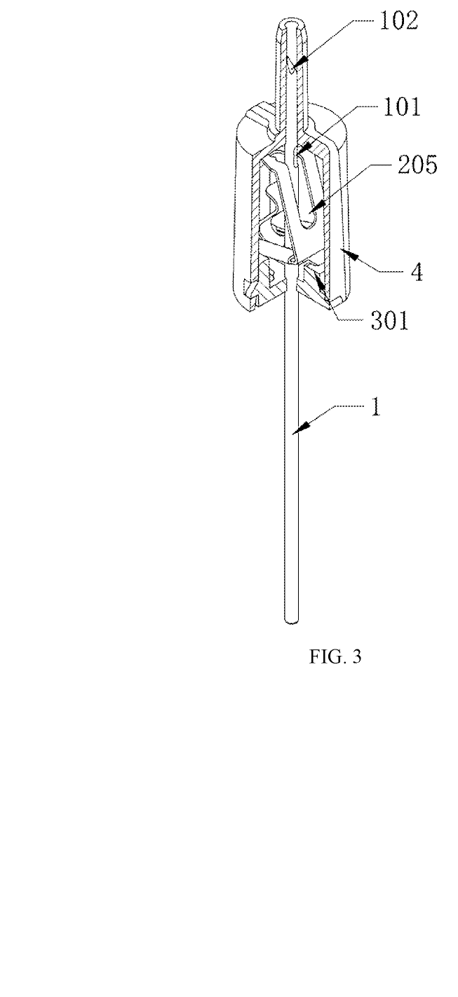

As shown in FIGS. 3, 5, 6, and 8, in the activated state, the blood flashback groove 101 in the puncture needle retreats to the height of the open guide hole 206 and moves from the opening 208 of the open guide hole 206 to the strip-shaped slot 205, the retainer elastic piece 2 tilts to the second side, the fixed portion 201 moves away from the surface of the limiting step 301, the bottom hole 202 tilts with the fixed portion 201, a wall of the bottom hole 202 exerts a cross torque on the outer periphery of the tube 1 of the puncture needle to hold the tube tightly, the resilient arm 203 still abuts against the tube 1 of the puncture needle and increases the torque at a contact point between the two as the retainer elastic piece tilts.

The aforementioned initial state includes an assembled state of each component of the needlestick injury prevention device for the medical puncture device and an usage state of the needlestick injury prevention device for the medical puncture device, and the activated state refers to a state where, after the needlestick injury prevention device for the medical puncture device is used, the puncture needle needs to be retracted, and the blood flashback groove provided in the outer periphery of the tube of the puncture needle is located in the open guide hole, which can activate the locking state of the puncture needle. In the initial state, it needs to ensure that the retainer elastic piece 2 is placed flat inside the sheath 4, thereby ensuring that the puncture needle extends smoothly out of the sheath 4. When the use is completed, in the activated state, the retainer elastic piece 2 tilts and locks the tube 1.

A relationship between the width at each position of the puncture needle and widths of the opening 208 of the open guide hole 206 and of the strip-shaped slot 205 can ensure that the tube 1 of the puncture needle is stably and coaxially arranged inside the bottom hole 202 and the open guide hole 206 in the initial state, and ensure that the retainer elastic piece 2 does not tilt prematurely. In the activated state, when the blood flashback groove 101 of the puncture needle is within the open guide hole 206, it can also ensure 360-degree tilt displacement for limited protection without blind spots, thereby improving the sensitivity and safety of the structure. In a preferred embodiment, the diameter of the open guide hole 206 is the same as that of the bottom hole 202, further improving the stability of the assembly between the tube 1 of the puncture needle and the retainer elastic piece 2 in the initial state.

In some embodiments, as shown in FIG. 12, the opening 208 between the open guide hole 206 and the strip-shaped slot 205 is provided with arc-shaped transition surfaces symmetrically arranged on its opposite edges. The arrangement of the arc-shaped transition surfaces at the edge of the opening 208 can stably guide the puncture tube 1 into the strip-shaped slot 205 smoothly in the activated state and guide the retainer elastic piece 2 to tilt.

In some embodiments, as shown in FIGS. 10 and 11, two reinforcing arms 207 are arranged at two ends of the fixed portion 201 between the support arm 204 and the resilient arm 203. Each of the reinforcing arms 207 has a greater thickness than other positions of the fixed portion 201. A first side shoulder 2071 is arranged at one side of each reinforcing arm 207 close to the support arm 204, and a second side shoulder 2072 is arranged at one side of each reinforcing arm 207 closed to the resilient arm 203. The fixed portion 201, the support arm 204, the resilient arm 203, and the reinforcing arms 207 are integrally formed. In a preferred embodiment, the reinforcing arms 207 are formed integrally by stamping and flanging, which is simple in process. Moreover, the thickness at the position of the reinforcing arms 207 is twice the thickness of other positions of the fixed portion 201.

In some embodiments, as shown in FIG. 11, the resilient arm 203 is generally S-shaped, including a second side arm that bends upward from one side of the fixed portion 201. The top of the second side arm bends to the second side to form a positioning portion 2031. In the initial state, a junction between the second side arm and the positioning portion elastically abuts against the outer periphery of the tube 1 of the puncture needle.

The second side arm includes a first connecting portion 2032 connected to the positioning portion 2031, and a second connecting portion 2034 connected to the fixed portion 201. A necked portion 2033 is arranged between the first connecting portion 2032 and the second connecting portion 2034 and gradually narrows from the first connecting portion 2032 to the second connecting portion 2034. The width of the first connecting portion 2032 is equal to the maximum width of the necked portion 2033, and the width of the second connecting portion 2034 is equal to a minimum width of the necked portion 2033.

In a preferred embodiment, the first side arm tilts toward the second side, and the second side arm tilts toward the first side. The connection point of the second side arm is located at an intersection between a plane where a side edge of the first side is located and the bottom hole 202. This ensures that when the puncture needle and the retainer elastic piece 2 are assembled, the resilient arm 203 is deformed after being expanded by the puncture needle and exerts an elastic retention force on the tube 1 of the puncture needle, ensuring that in the activated state, the tube 1 is smoothly pushed into the strip-shaped slot 205 when the tube 1 of the puncture needle at the position of the blood flashback groove 101 is within the open guide hole 206.

In some embodiments, as shown in FIG. 8, two pressing posts 404 are arranged on an inner wall of the sheath 4 at the second side. The distance between the two pressing posts 404 is equal to the distance between the two reinforcing arms 207, and the two pressing posts 404 are respectively arranged on top of the second side shoulders 2072 of the two reinforcing arms 207. After the retainer elastic piece 2 tilts, lower surfaces of the two pressing posts 404 on the inner wall of the sheath 4 can also abut against upper surface of the second side shoulders 2072 of the two reinforcing arms 207 even if the puncture needle is continued to be pushed outward, thereby achieving locking. At the same time, the inclined cross force generated at the position of the bottom hole 202 of the retainer elastic piece 2 on the outer periphery of the tube 1 locks the puncture tube 1, achieving a dual-insurance structure.

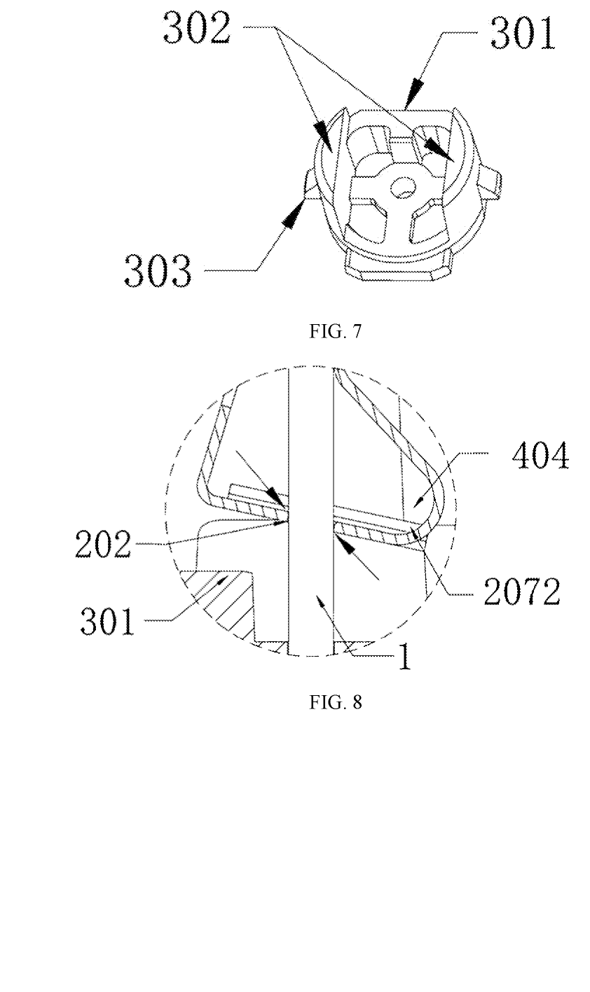

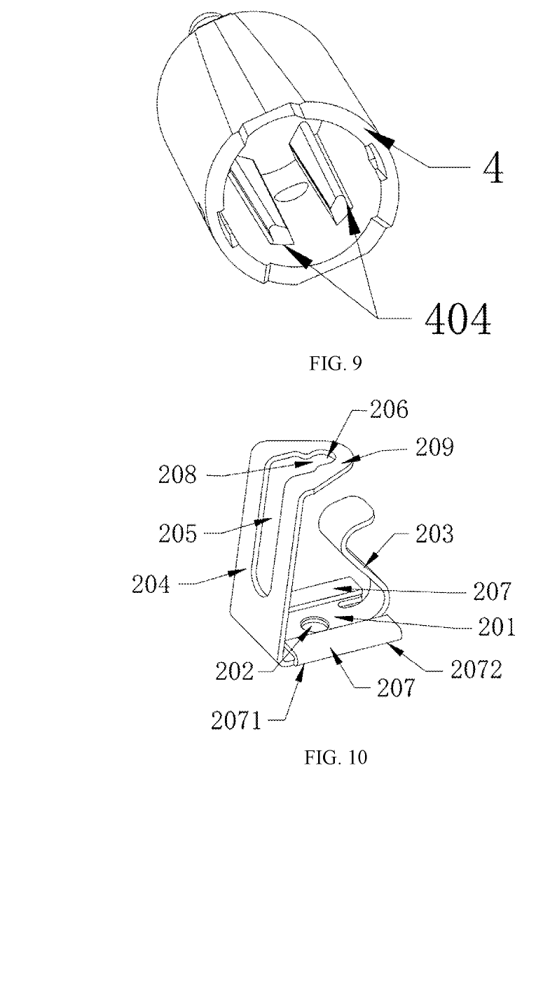

In some embodiments, as shown in FIG. 7, the base 3 is also provided with locking seats on both sides of the limiting step 301. A limiting groove 302 is provided at the top of each locking seat, and a latch 303 is arranged at the outer periphery of the locking seat. A catch 401 matching the latch 303 is provided in the outer periphery of the sheath 4, and the base 3 and the sheath 4 are fixed by the latch 303 and catch 401.

In some embodiments, as shown in FIG. 7, a first through hole is provided in the base 3, and a sleeve 402 is arranged at the top of the sheath 4. A puncture channel 403 is arranged in the sleeve 402, and the first through hole and the puncture channel 403 are coaxially arranged. In a preferred embodiment, the height between a needle tip 102 of the puncture needle and the blood flashback groove 101 is less than the height of the sleeve 402, which can ensure that in the activated state, the sleeve 402 completely encloses the needle tip 102 of the puncture needle, preventing the needle tip 102 from being exposed.

The present disclosure also discloses a medical injection and infusion device, including the aforementioned needlestick injury prevention device for the medical puncture device.

The present disclosure has various implementations. All technical solutions formed by equivalent transformations or transformations fall within the scope of protection of the present disclosure.

What is claimed is:

1. A needlestick injury prevention device for a medical puncture device, comprising a sheath, a puncture needle, and a retainer elastic piece, wherein the retainer elastic piece is arranged in the sheath, and the puncture needle is arranged between the retainer elastic piece and the sheath;
the sheath comprises a main body and a base arranged at a bottom of the main body; a limiting step is arranged on a first side of the base;
the puncture needle comprises a hollow tube, a needle tip located at one end of the tube, and a blood flashback groove provided in an outer periphery of the tube;
the retainer elastic piece comprises a fixed portion; a bottom hole is provided in the fixed portion, a support arm is arranged on a first side of the fixed portion, and an resilient arm is arranged on a second side of the fixed portion; and the support arm comprises a first side arm that bends upward from one side of the fixed portion and a guide portion that bends from a top of the first side arm and is parallel to the fixed portion, wherein a strip-shaped slot is provided in the support arm and communicates with the first side arm and the guide portion, an open guide hole is provided in the guide portion and provided in an end of the strip-shaped slot, an opening is formed in an end of the open guide hole that connects to the strip-shaped slot, and the open guide hole is coaxially arranged with the bottom hole;
a width of the tube of the puncture needle at a position of the blood flashback groove is less than a width of the opening of the open guide hole, the width of the opening of the open guide hole is less than an outer diameter of the tube of the puncture needle, and the outer diameter of the tube of the puncture needle is less than a width of the strip-shaped slot;
in an initial state, the first side of the fixed portion is placed flat on the limiting step, the tube of the puncture needle passes coaxially through the bottom hole and the open guide hole, the blood flashback groove is located at a top of the open guide hole, and the resilient arm elastically abuts against the tube of the puncture needle; and
in an activated state, the blood flashback groove in the puncture needle retreats to a height of the open guide hole and moves from the opening of the open guide hole to the strip-shaped slot, the retainer elastic piece tilts to the second side, the fixed portion moves away from a surface of the limiting step, the bottom hole tilts with the fixed portion, a wall of the bottom hole exerts a cross torque on the outer periphery of the tube of the puncture needle to hold the tube tightly, the resilient arm still elastically abuts against the tube of the puncture needle and a torque resulting from this abutment between the two increases as the retainer elastic piece tilts.

2. The needlestick injury prevention device for the medical puncture device according to claim 1, wherein the opening between the open guide hole and the strip-shaped slot is provided with arc-shaped transition surfaces symmetrically arranged on its opposite edges.

3. The needlestick injury prevention device for the medical puncture device according to claim 1, wherein two reinforcing arms are arranged at both ends of the fixed portion between the support arm and the resilient arm; each of the reinforcing arms has a greater thickness than other positions of the fixed portion; a first side shoulder is arranged at one side of each reinforcing arm close to the support arm, a second side shoulder is arranged at one side of each reinforcing arm close to the resilient arm; and the fixed portion, the support arm, the resilient arm, and the reinforcing arms are integrally formed.

4. The needlestick injury prevention device for the medical puncture device according to claim 1, wherein the resilient arm is generally S-shaped, comprising a second side arm that bends upward from one side of the fixed portion, a top of the second side arm bends toward the second side to form a positioning portion, and in the initial state, a junction between the second side arm and the positioning portionelastically abuts against the outer periphery of the tube of the puncture needle.

5. The needlestick injury prevention device for the medical puncture device according to claim 4, wherein the second side arm comprises a first connecting portion connected to the positioning portion, and a second connecting portion connected to the fixed portion; a necked portion is arranged between the first connecting portion and the second connecting portion and gradually narrows from the first connecting portion to the second connecting portion; and a width of the first connecting portion is equal to a maximum width of the necked portion, and a width of the second connecting portion is equal to a minimum width of the necked portion.

6. The needlestick injury prevention device for the medical puncture device according to claim 4, wherein the first side arm tilts toward the second side, and the second side arm tilts toward the first side, and the junction of the second side arm lies in a plane of a side edge on the first side, which plane intersects the bottom hole.

7. The needlestick injury prevention device for the medical puncture device according to claim 3, wherein two pressing posts are arranged on an inner wall of the sheath at the second side; a distance between the two pressing posts is equal to a distance between the two reinforcing arms; and the two pressing posts are respectively arranged on top of the second side shoulders of the two reinforcing arms.

8. The needlestick injury prevention device for the medical puncture device according to claim 1, wherein the base is provided with locking seats on both sides of the limiting step; a limiting groove is provided at a top of each locking seat; a latch is arranged at an outer periphery of the locking seat; and a catch matching the latch is arranged at an outer periphery of the sheath.

9. The needlestick injury prevention device for the medical puncture device according to claim 8, wherein a first through hole is provided in the base; a sleeve is arranged at a top of the sheath; a puncture channel is arranged in the sleeve; and the first through hole and the puncture channel are coaxially arranged.

10. A medical injection and infusion device, comprising the needlestick injury prevention device for the medical puncture device of claim 1.
